(19) United States
(12) Patent Application Publication
(10) Pub. No.: US 2023/0190474 A1
(43) Pub. Date: Jun. 22, 2023

(54) IMPLANTABLE INFLATABLE DEVICE

(71) Applicant: Boston Scientific Scimed, Inc., Maple Grove, MN (US)

(72) Inventors: Daragh Nolan, Youghal (IE); Thomas Sinnott, Wexford (IE); Richard Percy, Leamlara (IE); James Michael English, Cahir (IE); Barbara Belisa Soffiati, Clonmel (IE); Brian P. Watschke, Minneapolis, MN (US); Noel Smith, Windgap (IE); Eduardo Marcos Larangeira, Cork City (IE)

(21) Appl. No.: 18/068,127

(22) Filed: Dec. 19, 2022

Related U.S. Application Data
(60) Provisional application No. 63/265,813, filed on Dec. 21, 2021.

(51) Int. Cl. A61F 2/26

(52) U.S. Cl. CPC A61F 2/26

(57) ABSTRACT

An implantable inflatable device includes a fluid reservoir defining a cavity, an inflatable member, a pump assembly configured to transfer fluid between the fluid reservoir and the inflatable member, and a tubular member extending between the fluid reservoir and the pump assembly, the tubular member having a first end that is disposed within the cavity defined by the fluid reservoir.

### CROSS-REFERENCE TO RELATED APPLICATION

This application claims priority to U.S. Provisional Patent Application No. 63/265,813, filed on Dec. 21, 2021, entitled “IMPLANTABLE INFLATABLE DEVICE”, the disclosure of which is incorporated by reference herein in its entirety.

### TECHNICAL FIELD

This disclosure relates generally to bodily implants, and more specifically to bodily implants including an inflatable member, a fluid reservoir, and a pump.

### BACKGROUND

Implantable inflatable devices often include one or more pumps that regulate a flow of fluid between different portions of the implantable device to provide for inflation and deflation of one or more fluid fillable implant components of the device. For example, some implantable inflatable devices include an inflatable member, a fluid reservoir, and a pump. During the implantation process or during use of the device, air might get within the system. For example, air might get into the fluid reservoir and form air bubbles within the fluid reservoir. It is desirable to keep the air or air bubbles out of the inflatable member. For example, to allow the inflatable member to fully inflate with fluid, it may be desirable to keep the air or air bubbles out of the inflatable member. Accordingly, there is a need for an implantable inflatable device that functions to help retain any air or air bubbles within a portion of the device other than the inflatable member. For example, there is a need for an implantable inflatable device that functions to help retain air or air bubbles within the fluid reservoir.

### SUMMARY

According to an aspect, an implantable inflatable device includes a fluid reservoir defining a cavity; an inflatable member; a pump assembly configured to transfer fluid between the fluid reservoir and the inflatable member; and a tubular member extending between the fluid reservoir and the pump assembly, the tubular member having a first end that is disposed within the cavity defined by the fluid reservoir.

In some embodiments, the tubular member is operatively coupled to the fluid reservoir. In some embodiments, the tubular member has second end, the tubular member being operatively coupled to the fluid reservoir at a location along the tubular member, the location being disposed between the first end of the tubular member and the second end of the tubular member. In some embodiments, the tubular member has second end, the tubular member being operatively coupled to the fluid reservoir at a location along the tubular member, the location being disposed between the first end of the tubular member and the second end of the tubular member, the location being spaced from the first end of the tubular member. In some embodiments, the tubular member has second end, the tubular member being operatively coupled to the fluid reservoir at a location along the tubular member, the location being disposed between the first end of the tubular member and the second end of the tubular member, the location being spaced from the second end of the tubular member.

In some embodiments, the fluid reservoir is configured to retain fluid within the cavity defined by the fluid reservoir. In some embodiments, the fluid reservoir is configured to retain fluid within the cavity defined by the fluid reservoir, the first end portion of the tubular member being disposed within the cavity defined by the fluid reservoir such that it is configured to contact fluid that is disposed a distance from a sidewall of the reservoir. In some embodiments, the fluid reservoir is configured to retain fluid within the cavity defined by the fluid reservoir, the tubular member being operatively coupled to the reservoir at a location along the tubular member, the first end portion of the tubular member being disposed within the cavity defined by the fluid reservoir such that it is configured to contact fluid that is disposed a distance the location along the tubular member.

In some embodiments, the tubular member is a first tubular member, the inflatable device further comprising a second tubular member, the second tubular member extending between the pump assembly and the inflatable member.

In some embodiments, the pump assembly includes a pump member and a plurality of valve members.

In some embodiments, the inflatable member is configured to be placed in an inflated configuration and a deflated configuration. In some embodiments, the inflatable member is configured to be placed in an inflated configuration and a deflated configuration, the inflatable member being larger when in the inflated configuration. In some embodiments, the inflatable member is a first inflatable member, the inflatable device further comprising a second inflatable member. In some embodiments, the inflatable member is a first inflatable member, the inflatable device further comprising a second inflatable member, the second inflatable member being operatively coupled to the pump assembly.

In some embodiments, the fluid reservoir includes a sidewall having an inner surface and an outer surface opposite the inner surface.

In some embodiments, an implantable inflatable device includes a fluid reservoir having a sidewall defining a cavity and an extension member extending from the sidewall into the cavity defined by the fluid reservoir; an inflatable member; a pump assembly configured to transfer fluid between the fluid reservoir and the inflatable member; and a tubular member extending between the fluid reservoir and the pump assembly.

In some embodiments, the tubular member is operatively coupled to the fluid reservoir.

In some embodiments, the extension member forms a closed shape.

In some embodiments, the sidewall includes an inner surface and an outer surface, the extension member being coupled to and extending from the inner surface of the sidewall. In some embodiments, the sidewall includes an inner surface and an outer surface, the inner surface defining the cavity, the extension member begin coupled to and extending from the inner surface of the sidewall.

In some embodiments, the fluid reservoir is configured to retain fluid within the cavity defined by the fluid reservoir.

In some embodiments, the tubular member is a first tubular member, the inflatable device further comprising a second tubular member, the second tubular member extending between the pump assembly and the inflatable member.

In some embodiments, a method includes coupling a pump assembly to an inflatable member via a first tubular member; and coupling the pump assembly to a fluid reservoir via a second tubular member such that an end portion of the second tubular member is disposed within a cavity defined by the fluid reservoir.

In some embodiments, the coupling the pump assembly to the fluid reservoir includes coupling the fluid reservoir to an outer surface of the second tubular member at a location, the location being disposed between the first end portion of the second tubular member and a second end portion of the second tubular member. In some embodiments, the coupling the pump assembly to the fluid reservoir includes coupling the fluid reservoir to an outer surface of the second tubular member at a location, the location being disposed a distance from the first end portion of the second tubular member and a distance from a second end portion of the second tubular member.

In some embodiments, the fluid reservoir is configured to retain fluid within the cavity defined by the fluid reservoir, the coupling the pump assembly to the fluid reservoir includes coupling the fluid reservoir to the second tubular member such that the end portion of the tubular member is disposed within the cavity such that it is configured to contact fluid that is disposed a distance from a sidewall of the reservoir.

### BRIEF DESCRIPTION OF THE DRAWINGS

FIG. 1 is a schematic illustration of an implantable inflatable device according to an aspect.

FIG. 2 illustrates an implantable inflatable device according to an aspect.

FIG. 3 is a perspective view of the fluid reservoir of the device of FIG. 2.

FIGS. 4 and 5 are cross-sectional views of the fluid reservoir of FIG. 3.

FIG. 6 is a perspective view of a reservoir according to an aspect.

FIGS. 7 and 8 are cross-sectional views of the reservoir of FIG. 6.

FIG. 9 is a flow chart of a method according to an aspect.

### DETAILED DESCRIPTION

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to variously employ the implementations in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the implementations are directed to bodily implants. The term patient or user may hereinafter be used for a person who benefits from the medical device or the methods disclosed in the present disclosure. For example, the patient can be a person whose body is implanted with the medical device or the method disclosed for operating the medical device by the present disclosure.

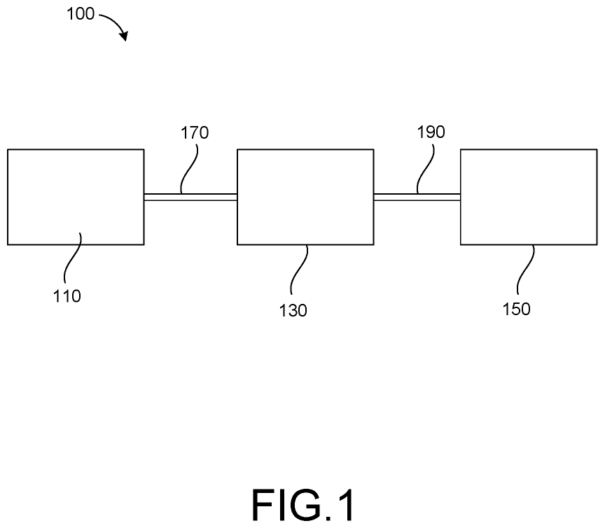

FIG. 1 is a schematic illustration of an implantable inflatable device 100. The device 100 includes a fluid reservoir 110, a pump assembly 130, and an inflatable member 150. The fluid reservoir 110 is operatively or fluidically coupled to the pump assembly 130 via connection member 170. The connection member 170 may be a tubular member such as a kink resistant tubing (KRT). In other implementations, the fluid reservoir 110 is operatively or fluidically coupled to the pump assembly 130 via a different mechanism. Similarly, the inflatable member 150 is operatively or fluidically coupled to the pump assembly 130 via connection member 190. Connection member 190 may be a tubular member such as a kink resistant tubing (KRT). In other implementations, the inflatable member 150 is operatively or fluidically coupled to the pump assembly 130 via a different mechanism.

The implantable inflatable device 100 may be configured to be implanted into a body of a patient. For example, in some embodiments, the implantable inflatable device 100 is a penile implant. In such embodiments, the inflatable member 150 may be implanted into the corpus cavernosae of the patient or user, the fluid reservoir 110 may be implanted in the abdomen or pelvic cavity of the user (e.g., the fluid reservoir 110 may be implanted in the lower portion of the user's abdominal cavity or the upper portion of the user's pelvic cavity), and the pump assembly 130 may be implanted in the scrotum of the user. In other embodiments, the implantable inflatable device 100 is implanted into a different portion of the body of the patient and/or is implanted for a different purpose. For example, in some embodiments, the implantable inflatable device 100 may be an artificial sphincter, such as an artificial urinary sphincter.

The pump assembly 130 may include a pump or more than one pump that is configured pump fluid into the inflatable member 150 during an inflation cycle. In some examples, the pump or pumps maybe be manually controlled by the user or may be mechanically and/or programmatically controlled by a controller.

The inflatable member 150 may be capable of expanding upon the injection of fluid into a cavity of the inflatable member 150. For instance, upon injection of the fluid into the inflatable member 150, the inflatable member 150 may increase its length and/or width, as well as increase its rigidity. In some examples, the inflatable member 150 may include a pair of inflatable cylinders or at least two cylinders, e.g., a first cylinder member and a second cylinder member. The volumetric capacity of the inflatable member 150 may depend on the size of the inflatable cylinders.

The fluid reservoir 110 may include a container having an internal cavity or chamber configured to hold or house fluid that is used to inflate the inflatable member 150. The volumetric capacity of the fluid reservoir 110 may vary. In some examples, the volumetric capacity of the fluid reservoir 110 may be 3 to 150 cubic centimeters. In some examples, the fluid reservoir 110 is constructed from the same material as the inflatable member 150. In other examples, the fluid reservoir 110 is constructed from a different material than the inflatable member 150. In some examples, the fluid reservoir 110 contains a larger volume of fluid than the inflatable member 150.

In some embodiments, the fluid reservoir 110 is configured to hold or retain air or air bubbles that may get into the device 100. Such bubbles may develop during the implantation process or during use of the device 100. For example, in some embodiments, the fluid reservoir 110 is configured to retain bubbles within the cavity of the fluid reservoir 110 that develop within the device 100. In such embodiments, the bubbles are not advanced or moved to the pump assembly 130 or the inflatable member 150 when the pump assembly 130 draws fluid from the fluid reservoir and passes such fluid to the inflatable member 150.

In some embodiments, the connection member 170 includes an end portion that is disposed within the cavity of the fluid reservoir 110. In such embodiments, the sidewall or outer surface of the end portion of the connection member 170 helps retain the bubbles within the cavity of the fluid reservoir 110. Accordingly, the bubbles are not advanced or moved to the pump assembly 130 or the inflatable member 150 when the pump assembly 130 draws fluid from the fluid reservoir and passes such fluid to the inflatable member 150. For example, in some embodiments, the bubbles may be attached to, adhered to, or otherwise engage with the sidewall or outer surface of the end portion of the connection member 170 when fluid is drawn from the fluid reservoir 110 and into the connection member 170.

In other embodiments, the fluid reservoir 110 includes a projection member that extends into the cavity of the fluid reservoir 110. In such embodiments, the projection member is configured to help retain the bubbles within the fluid reservoir 110.

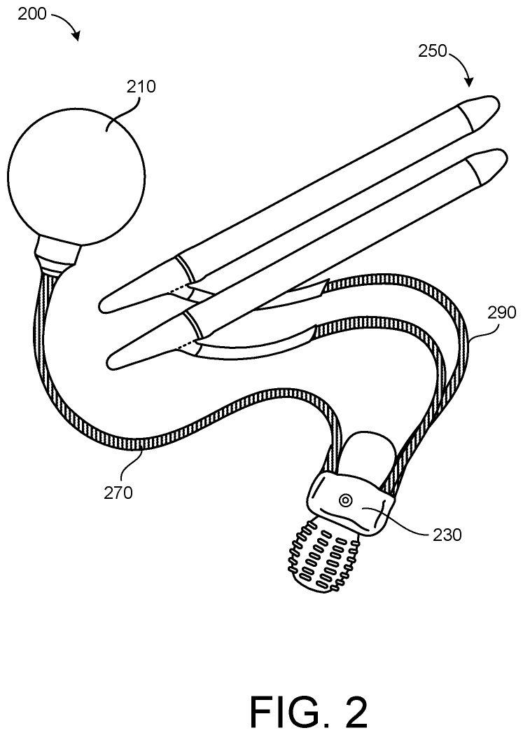

FIG. 2 illustrates an inflatable penile prosthesis 200 having a pump assembly 230 according to an aspect. The pump assembly 230 may include valves and may include manually actuated pump bulb or may include an electronically controlled pump. The penile prosthesis 200 may include one or more inflatable members or inflatable cylinders 250. In the illustrated embodiment, the prosthesis 200 includes a pair of inflatable cylinders 250. The inflatable cylinders 250 are configured to be implanted in a penis. For example, one of the inflatable cylinders 250 may be disposed on one side of the penis, and the other inflatable cylinder 250 may be disposed on the other side of the penis. Each inflatable cylinder 250 may include a first end portion, a cavity or inflation chamber, and a second end portion having a rear tip. The first end portion of the inflatable cylinder 250 may be at least partially disposed within the crown portion of the penis. The second end portion may be implanted into the patient's pubic region with the rear tip proximate the pubic bone.

The pump assembly 230 may be implanted into the patient's scrotum. A pair of conduit connectors 290 may attach the pump assembly 230 to the inflatable cylinders 250 such that the pump assembly 230 is in fluid communication with the inflatable cylinders 250. Also, the pump assembly 230 may be in fluid communication with a fluid reservoir 210 via a connection member or a conduit connector 270. The fluid reservoir 210 may be implanted into the user's abdomen.

In order to implant the inflatable cylinders 250, the surgeon first prepares the patient. The surgeon often makes an incision in the penoscrotal region, e.g., where the base of the penis meets with the top of the scrotum. From the penoscrotal incision, the surgeon may dilate the patient's corpus cavernosae to prepare the patient to receive the inflatable cylinders 250. The corpus cavernosum is one of two parallel columns of erectile tissue forming the dorsal part of the body of the penis, e.g., two slender columns that extend substantially the length of the penis. The surgeon will also dilate two regions of the pubic area to prepare the patient to receive the second end portion. The surgeon may measure the length of the corpora cavernosae from the incision and the dilated region of the pubic area to determine an appropriate size of the inflatable cylinders 250 to implant.

After the patient is prepared, the penile prosthesis 200 is implanted into the patient. The tip of the first end portion of each inflatable cylinder 250 may be attached to a suture. The other end of the suture may be attached to a needle member (e.g., Keith needle). The needle member is inserted into the incision and into the dilated corpus cavernosum. The needle member is then forced through the crown of the penis. The surgeon tugs on the suture to pull the inflatable cylinder 250 into the corpus cavernosum. This is done for each inflatable cylinder 250 of the pair. Once the inflation chamber is in place, the surgeon may remove the suture from the tip. The surgeon then inserts the second end portion. The surgeon inserts the rear end of the inflatable cylinder 250 into the incision and forces the second end portion toward the pubic bone until each inflatable cylinder 250 is in place.

In some embodiments, the pump assembly 230 is a manual pump. In such embodiments, a pump bulb of the pump assembly 230 may be squeezed or depressed by the user in order to facilitate the transfer of fluid from the fluid reservoir 210 to the inflatable cylinders 250. For example, in the inflation mode, while the user is operating the pump bulb, the pump bulb may receive the fluid from the fluid reservoir 210, and then output the fluid to the inflatable cylinders 250. When the user switches to the deflation mode, at least some of the fluid can automatically be transferr150

ed back to the fluid reservoir 210 (due to the difference in pressure from the inflatable cylinders 250 to the fluid reservoir 210). Then, the user may squeeze the inflatable cylinders 250 to facilitate the further transfer of fluid through the pump assembly 230 to the fluid reservoir 210.

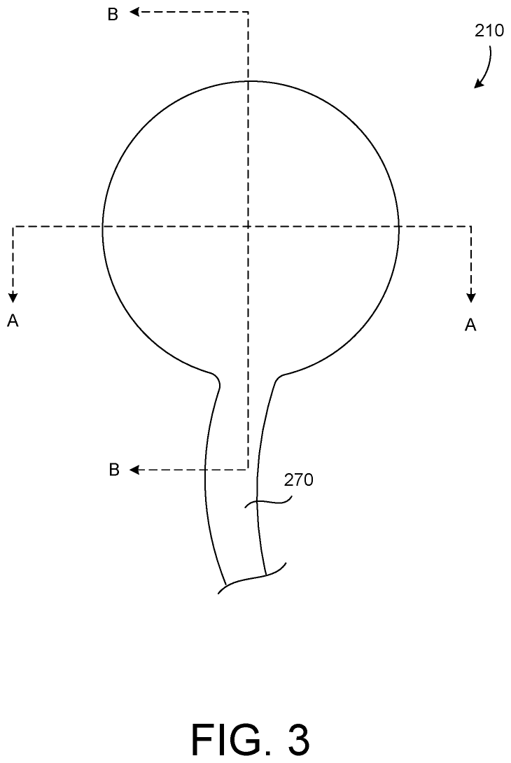

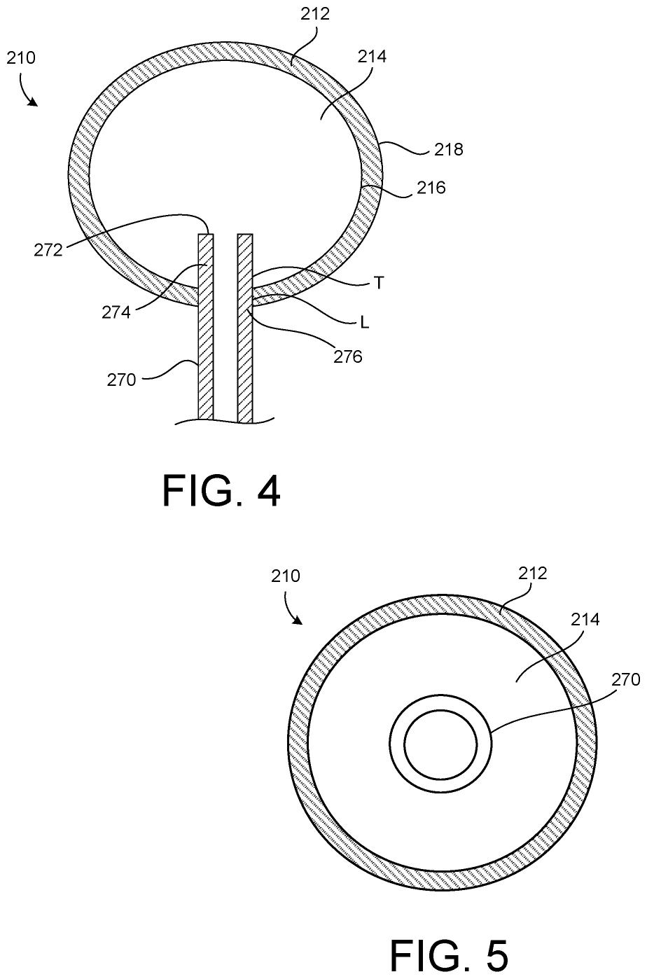

FIG. 3 is a perspective view of the fluid reservoir 210 and a portion of the connection member 270. FIG. 4 is a cross-sectional view of the fluid reservoir 210 and the portion of the connection member 270 taken along line B-B of FIG. 3. FIG. 5 is a cross-sectional view of the fluid reservoir 210 and the portion of the connection member 270 taken along line A-A of FIG. 3.

The fluid reservoir 210 has a sidewall 212. The sidewall 212 defines a cavity 214 and has an inner surface 216 and an outer surface 218. The outer surface 218 is disposed opposite the inner surface 216. The connection member or tubular member 270 is operatively coupled to the fluid reservoir 210 and provides a fluidic coupling between the pump assembly 230 and the fluid reservoir 210. Although the tubular member 270 is illustrated as being a cylindrical tube, in other embodiments, the tubular member is of a different shape. For example, in some embodiments, the tubular member has a square, rectangular, oval, or other shape.

In the illustrated embodiment, the fluid reservoir 210 is coupled to the tubular member 270 such that a portion of the tubular member 270 is disposed within the cavity 214. Specifically, an end 272 of the tubular member 270 is disposed within the cavity 214. In other words, the end portion 274 of the tubular member 270 extends from the inner surface 216 of the sidewall 212 into the cavity 214. In some embodiments, the end portion 274 extends a few centimeters into the cavity 214. In other embodiments, the end portion 274 extends more than a few centimeters into the cavity 214.

In the illustrated embodiment, the sidewall 212 of the fluid reservoir 210 is coupled to an outer surface 276 of the tubular member 270 at a location L along the tubular member 270. The coupling location L is disposed between the end 272 of the tubular member 270 and the end portion of the tubular member 270 that is operatively coupled to the pump assembly 230. Specifically, the coupling location L is disposed a distance from both the end 272 and the end of the tubular member 270 that is coupled to the pump assembly 230.

In use, when the pump assembly 230 is activated to move fluid from the fluid reservoir 210 to the inflatable members 250, fluid will be pulled into the tubular member 270. Specifically, the fluid that is pulled from a location within the cavity 214 that is disposed apart from the sidewall 212 of the fluid reservoir 210. Accordingly, any bubbles that might be located near the sidewall 212 of the fluid reservoir 210 will not be pulled into the tubular member 270 and passed along to the inflatable members 250. For example, small bubbles such as microbubbles which tend to adhere to the surface (the inner surface 216) of the sidewall 212 will not be pulled into the tubular member 270. Specifically, such small bubbles may be pulled along the inner surface 216 of the sidewall 212 but will ultimately be trapped at location T within the cavity 214 (the location near the intersection of the sidewall 212 and the tubular member 270).

In some embodiments, the fluid reservoir 210 and the tubular member 270 may be formed of silicone (such as 2-part liquid silicone), rubber (such as high consistency rubber), silicone dispersions, or in a fluorosilicone. In some embodiments, the materials that form the fluid reservoir 210 and the tubular member 270 may have non-stick properties. Such non-stick features may help prevent sides (such as portions of the inner surface) of the fluid reservoir 210 from sticking together during use. The fluid reservoir 210 and the tubular member 270 may be formed using know processes such as molding (including liquid silicone molding), extrusion, or dip casting.

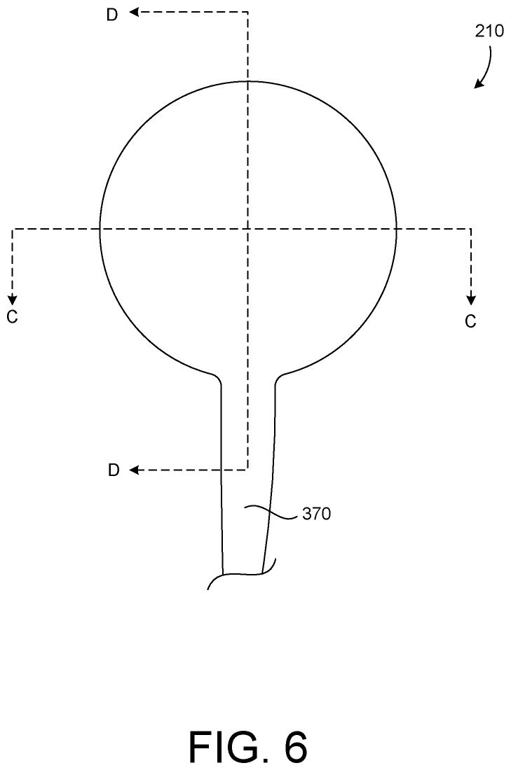

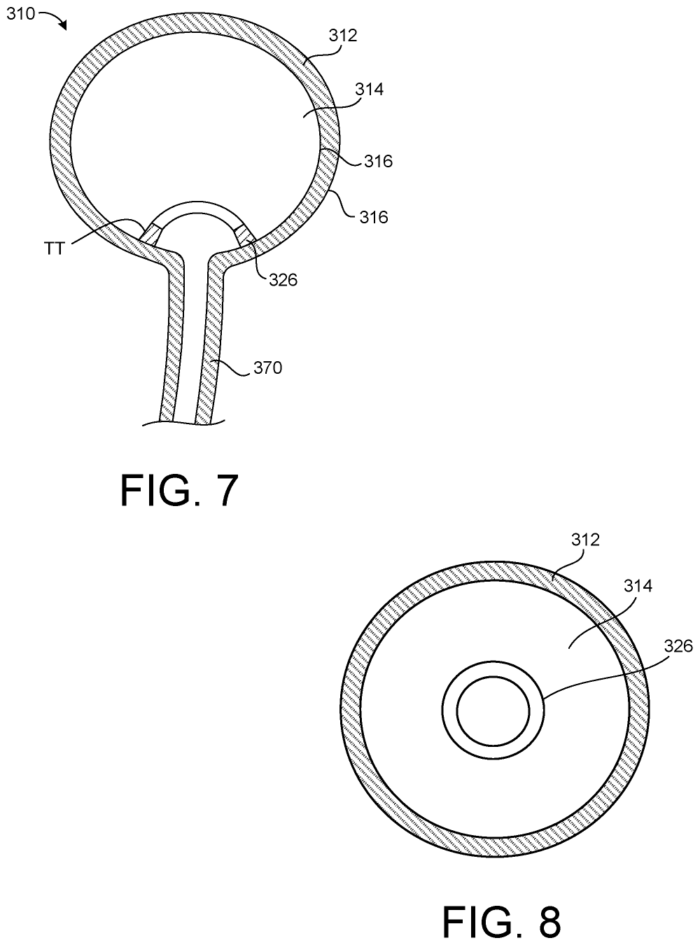

FIGS. 6-8 illustrate a fluid reservoir 310 according to another embodiment. FIG. 6 is a perspective view of the fluid reservoir 310 and a portion of the connection member 370. FIG. 7 is a cross-sectional view of the fluid reservoir 310 and the portion of the connection member 370 taken along line D-D of FIG. 6. FIG. 8 is a cross-sectional view of the fluid reservoir 310 and the portion of the connection member 370 taken along line C-C of FIG. 6.

The fluid reservoir 310 has a sidewall 212. The sidewall 312 defines a cavity 314 and has an inner surface 316 and an outer surface 318. The outer surface 318 is disposed opposite the inner surface 316. The connection member or tubular member 370 is operatively coupled to the fluid reservoir 310 and provides a fluidic coupling between the pump assembly and the fluid reservoir 310. Although the tubular member 370 is illustrated as being a cylindrical tube, in other embodiments, the tubular member is of a different shape. For example, in some embodiments, the tubular member has a square, rectangular, oval, or other cross-sectional shape.

In the illustrated embodiment, the fluid reservoir 310 includes an extension or projection member 326. The extension or projection member 326 is coupled to the inner surface 316 of the sidewall 312. The extension or projection member 326 extends from the inner surface 316 of the sidewall 312 and into the cavity 314 defined by the sidewall 312. In the illustrated embodiment, the extension or projection member 326 is located near or proximate the location where the connection member or tubular member 370 is coupled to the fluid reservoir 310. In some embodiment, the extension or projection member 326 is formed separately from the sidewall 312 of the fluid reservoir 310 and is later coupled to the sidewall. In other embodiments, the extension or projection member 326 is formed with the sidewall 312. For example, the extension or projection member 326 may be unitarily or monolithically formed with the sidewall 312 (or made from one piece of material with the sidewall).

As best illustrated in FIG. 8, in the illustrated embodiment, the extension or projection member 326 extends around an entire circumference of the inner surface 316 and forms a circle. In other embodiments, the extension or projection member 326 may form a different shape, such as an oval, square, or rectangle. Also, in other embodiments, the extension or projection member does not extend about an entire circumference of the inner surface.

In use, when the pump assembly is activated to move fluid from the fluid reservoir 310 to the inflatable members, fluid will be pulled into the tubular member 370. Bubbles that might be located near the sidewall 312 of the fluid reservoir 310 will not be pulled into the tubular member 370 and passed along to the inflatable members. For example, small bubbles such as microbubbles which tend to adhere to the surface (the inner surface 316) of the sidewall 312 will not be pulled into the tubular member 370. Specifically, such small bubbles may be pulled along the inner surface 316 of the sidewall 312 but will ultimately be trapped at location TT within the cavity 314 (the location near the intersection of the sidewall 312 and the extension or projection member 326).

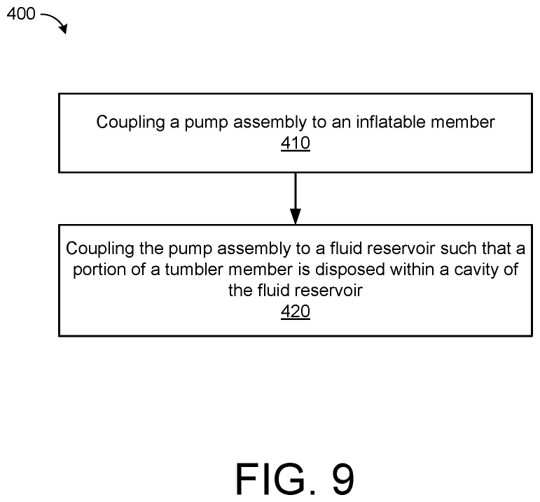

FIG. 9 is a flow chart of a method 400 of forming an implant. The method 400 includes at 410 coupling a pump assembly to an inflatable member via a first tubular member and at 420 coupling the pump assembly to a fluid reservoir via a second tubular member such that an end portion of the second tubular member is disposed within a cavity defined by the fluid reservoir.

In some embodiments, the method includes coupling the fluid reservoir to an outer surface of the second tubular member at a coupling location. The coupling location is disposed between the first end portion of the second tubular member and a second end portion of the second tubular member.

In some embodiments, the method includes coupling the fluid reservoir to an outer surface of the second tubular member at a coupling location. The coupling location is disposed a distance from the first end portion of the second tubular member and a distance from a second end portion of the second tubular member.

In some embodiments, the fluid reservoir is configured to retain fluid within the cavity defined by the fluid reservoir. The method includes coupling the fluid reservoir to the second tubular member such that the end portion of the tubular member is disposed within the cavity such that it is configured to contact fluid that is disposed a distance from a sidewall of the fluid reservoir.

While certain features of the described implementations have been illustrated as described herein, many modifications, substitutions, changes, and equivalents will now occur to those skilled in the art. It is, therefore, to be understood that the appended claims are intended to cover all such modifications and changes as fall within the scope of the embodiments.

What is claimed is:

1. An implantable inflatable device, comprising:
a fluid reservoir defining a cavity;
an inflatable member;
a pump assembly configured to transfer fluid between the fluid reservoir and the inflatable member; and
a tubular member extending between the fluid reservoir and the pump assembly, the tubular member having a first end that is disposed within the cavity defined by the fluid reservoir.

2. The inflatable device of claim 1, wherein the tubular member is operatively coupled to the fluid reservoir.

3. The inflatable device of claim 1, wherein the tubular member has second end, the tubular member being operatively coupled to the fluid reservoir at a location along the tubular member, the location being disposed between the first end of the tubular member and the second end of the tubular member.

4. The inflatable device of claim 1, wherein the tubular member has second end, the tubular member being operatively coupled to the fluid reservoir at a location along the tubular member, the location being disposed between the first end of the tubular member and the second end of the tubular member, the location being spaced from the first end of the tubular member.

5. The inflatable device of claim 1, wherein the tubular member has second end, the tubular member being operatively coupled to the fluid reservoir at a location along the tubular member, the location being disposed between the first end of the tubular member and the second end of the tubular member, the location being spaced from the second end of the tubular member.

6. The inflatable device of claim 1, wherein the fluid reservoir is configured to retain fluid within the cavity defined by the fluid reservoir.

7. The inflatable device of claim 1, wherein the fluid reservoir is configured to retain fluid within the cavity defined by the fluid reservoir, the first end of the tubular member being disposed within the cavity defined by the fluid reservoir such that it is configured to contact fluid that is disposed a distance from a sidewall of the reservoir.

8. The inflatable device of claim 1, wherein the fluid reservoir is configured to retain fluid within the cavity defined by the fluid reservoir, the tubular member being operatively coupled to the reservoir at a location along the tubular member, the first end of the tubular member being disposed within the cavity defined by the fluid reservoir such that it is configured to contact fluid that is disposed a distance the location along the tubular member.

9. The inflatable device of claim 1, wherein the tubular member is a first tubular member, the inflatable device further comprising a second tubular member, the second tubular member extending between the pump assembly and the inflatable member.

10. An implantable inflatable device, comprising:
a fluid reservoir having a sidewall defining a cavity and an extension member extending from the sidewall into the cavity defined by the fluid reservoir;
an inflatable member;
a pump assembly configured to transfer fluid between the fluid reservoir and the inflatable member; and
a tubular member extending between the fluid reservoir and the pump assembly.

11. The inflatable device of claim 10, wherein the tubular member is operatively coupled to the fluid reservoir.

12. The inflatable device of claim 10, wherein the extension member forms a closed shape.

13. The inflatable device of claim 10, wherein the sidewall includes an inner surface and an outer surface, the extension member being coupled to and extending from the inner surface of the sidewall.

14. The inflatable device of claim 10, wherein in the sidewall includes an inner surface and an outer surface, the inner surface defining the cavity, an extension member being coupled to and extending from the inner surface of the sidewall.

15. The inflatable device of claim 10, wherein the fluid reservoir is configured to retain fluid within the cavity defined by the fluid reservoir.

16. The inflatable device of claim 10, wherein the tubular member is a first tubular member, the inflatable device further comprising a second tubular member, the second tubular member extending between the pump assembly and the inflatable member.

17. A method, comprising:
coupling a pump assembly to an inflatable member via a first tubular member; and
coupling the pump assembly to a fluid reservoir via a second tubular member such that an end portion of the second tubular member is disposed within a cavity defined by the fluid reservoir.

18. The method of claim 17, wherein the coupling the pump assembly to the fluid reservoir includes coupling the fluid reservoir to an outer surface of the second tubular member at a location, the location being disposed between the first end portion of the second tubular member and a second end portion of the second tubular member.

19. The method of claim 17, wherein the coupling the pump assembly to the fluid reservoir includes coupling the fluid reservoir to an outer surface of the second tubular member at a location, the location being disposed a distance from the first end portion of the second tubular member and a distance from a second end portion of the second tubular member.

20. The method of claim 17, wherein the fluid reservoir is configured to retain fluid within the cavity defined by the fluid reservoir, the coupling the pump assembly to the fluid reservoir includes coupling the fluid reservoir to the second tubular member such that the end portion of the tubular member is disposed within the cavity such that it is configured to contact fluid that is disposed a distance from a sidewall of the fluid reservoir.
